(19) United States
(12) Patent Application Publication
(10) Pub. No.: US 2018/0245066 A1
(43) Pub. Date: Aug. 30, 2018

(54) sgRNA and knockout method of human RSPO2 gene targeted with CRISPR-Cas9 specificity and application thereof

(71) Applicant: The First Hospital of Jiaxing, Jiaxing (CN)

(72) Inventors: Ming Yao, Jiaxing (CN); Linghua Yu, Jiaxing (CN)

(73) Assignee: The First Hospital of Jiaxing

(21) Appl. No.: 15/961,886

(22) Filed: Apr. 25, 2018

(30) Foreign Application Priority Data
Dec. 7, 2017 (CN) 201711288951.3

(51) Int. Cl. C12N 15/10; C12N 15/11; C12N 9/22

(52) U.S. Cl. CPC C12N 15/102; C12N 2310/20; C12N 9/22; C12N 15/11

(57) ABSTRACT

A method for knocking out a human RSPO2 gene targeted with CRISPR-Cas9 specificity includes steps of: 1) designing the sgRNA of the human RSPO2 gene targeted; and 2) constructing a CRISPR-Cas9 recombinant lentivirus vector for knocking out the RSPO2 gene. A method for preparing a lentiviral-packaged system for knocking out a human RSPO2 gene targeted with CRISPR-Cas9 specificity includes steps of: 1) designing the sgRNA of the human RSPO2 gene targeted; 2) constructing a CRISPR-Cas9 recombinant lentivirus vector for knocking out the RSPO2 gene; and 3) processing the CRISPR-Cas9 recombinant lentivirus vector for knocking out the sgRNA of the human RSPO2 gene with lentiviral packaging, so as to obtain the lentiviral-packaged system.

### CROSS REFERENCE OF RELATED APPLICATION

The present invention claims priority under 35 U.S.C. 119(a-d) to CN 201711288951.3, filed Dec. 7, 2017.

### BACKGROUND OF THE PRESENT INVENTION

### Field of Invention

The present invention relates to a biotechnology field, and more particularly to a sgRNA and a knockout method of a human RSPO2 gene targeted with CRISPR-Cas9 specificity and application thereof.

### Description of Related Arts

The CRISPR-Cas9 (Clustered Regularly Interspaced Short Palindromic Repeats associated) widely exists in bacteria and archaea, which is a RNA-guided heritable adaptive immunity system. CRISPR (Clustered Regularly Interspaced Short Palindromic Repeats) is composed of highly conserved repeats and multiple spacers which are arranged in order. A length of the repeats is 21-48 bp. The repeats is spaced by spacers of 26-72 bp. Cas9 (CRISPR associated) is a double stranded DNA nuclease which comprises two domains: 1) HNH-like domain cuts the DNA strand complementary to the crRNA (CRISPR RNA); 2) RuvC-like domain cuts non-complementary strand. The basic mechanism of the CRISPR-Cas9 is as follow 1) transcribing and processing the CRISPR sequence into crRNA; 2) recruiting Cas9 protein by tracrRNA (trans-activating crRNA); 3) matching the spacers of crRNA with the neighboring target of PAM (Protospacer Adjacent Motif) to instruct Cas9 protein to cut the target. The double-cleavage activity of Cas9 protein is activated to cause double-stranded breaks (DSB) at the target site, and the broken double-stranded DNA is amplified by non-homologous end joining (NHEJ) or homologous recombination homology-directed repair (HDR). The repaired DNA inhibits the expression of the gene due to the frameshift mutation caused by the random insertion or deletion of the base, which realizes targeted knock-out of the gene at the DNA level.

Editing the target sequence by CRISPR specificity is realized by the complementary identification of the target sequence. The tracrRNA and crRNA are expressed as a single guide RNA (sgRNA). The CRISPR-Cas9 system is simplified as Cas9 protein and sgRNA, which is easy to construct and with high efficiency and low cost. The simplified CRISPR-Cas9 system is a most suitable choice for gene editing. To design an accurate target sequence sgRNA is the key technology of the CRISPR-Cas9 system.

Liver fibrosis is a reversible wound-healing response to a variety of insults. With chronic liver injury, this wound-healing process is presented as a progressive substitution of the functional parenchyma by scar tissue. The pathological characteristics are that various compositions, mainly collagen, of the extracellular matrix are synthesized and increased while the degradation is relatively insufficient and the interlobular septa are not formed. Further development leads to cirrhosis. The liver fibrosis is reversible. A prevention and early intervention to the liver fibrosis is the best practice to stable the condition and prevent the liver fibrosis from developing into cirrhosis and liver cancer.

HSC (Hepatic Stellate Cell) is the primary cell type responsible for extracellular matrix synthesis and degradation. HSC activation and phenotypic switch to a myofibroblast-like cell is the central event of liver fibrogenesis. The activation of the hepatic stellate cell is regulated by multiple signal pathways. Research shows that the Wnt signal pathway affects a competence of the hepatic stellate cell and the blockage of the Wnt signal pathway suppresses the hepatic stellate cell proliferation and induces the hepatic stellate cell death. Because the Wnt signal pathway participates in various biological processes including the differentiation and maintenance of the cell form and function, immunity, and cell carcinogenesis and death, a direct blockage of the Wnt signal path may causes adverse biological effects. RSPO2 (R-spondin2) is an important newly discovered regulation factor of the Wnt signal factor, which is able to activate and enhance the Wnt/β-catenin signal pathway and play an important role in tissue differentiation, organogenesis and diseases.

To regulate the competence of hepatic stellate cell without blocking the important signal pathway such as Wnt directly is a pressing problem needs to be solved.

RSPO2 antibodies are conventionally used in the art to treat fibrosis (as disclosed in Chinese patent application 201580049993.4). However, the use of RSPO antibodies for target gene therapy is limited by many technical factors: (1) antibodies can only temporarily block the target receptor; (2) it is not easy to develop effective antibodies; (3) it is not possible to block multiple inhibitory receptor; and (4) it is only effective to extracellular targets.

### SUMMARY OF THE PRESENT INVENTION

An object of the present invention is to provide a method for knocking out human RSPO2 gene with CRISPR-Cas9 specificity, and repress Wnt/β-catenin signal pathway to convert activated hepatic stellate cell to quiescent state or apoptosis, for effectively promoting recovery of liver fibrosis. The present invention designs and synthesizes sgRNA of a specific target RSPO2, wherein the sgRNA is connected to the lentiviral vector and is packaged as lentivirus, so as to achieve stable intracellular transcription of the sgRNA for long-term inhibition of target gene expression.

The technical solution to solve the problem is as follows:

Firstly, a method for knocking out a human RSPO2 gene with CRISPR-Cas9 specificity comprises steps of:

I. designing a sgRNA (single guide RNA) specifically targeted human RSPO2 gene

1. designing the sgRNA of the targeted human RSPO2 gene, wherein the sgRNA satisfies following conditions:

(1) a length of the sgRNA is 20 nucleotide sequences;

(2) a target of sgRNA on the RSPO2 gene locates in an exon thereof;

(3) preferably, the sgRNA target on the RSPO2 gene locates in a functional domain thereof;

(4) 5′-NGG is selected for PAM of a target sequence;

(5) preferably, a sgRNA target sequence is started at G to ensure an effective U6 promoter of a vector; and

(6) a format of the sgRNA target sequence is as follows:

5′-G-(19N)-NGG-3′ (the sgRNA target sequence starts at G)

or 5′-(20N)-NGG-3′ (the sgRNA target sequence doesn't start at G)

wherein, 19N or 20N refers to 19 or 20 nucleotide sequences of the sgRNA target.

2. selecting the sgRNA of the targeted human RSPO2 gene, satisfying following conditions:

(1) a BLAST (Basic Local Alignment Search Tool) is adopted in a NCBI (National Center for Biotechnology Information) database to ensure a uniqueness of the sgRNA target sequence which is not paralogous with gene sequences other than the human RSPO2 gene;

(2) the sgRNA target locates in DHSs (DNase I hypersensitive sites);

(3) there is a certain distance between the sgRNA target and a start cordon (ATG); and

(4) an off-target rate is low.

Five target sequences are selected, as shown in Table 1.

TABLE 1
target sequences corresponding to different
locus of RSPO2 gene

| target | | |
|---|---|---|
| number | target sequence | PAM |
| 1 | 5′-TTGTCTTGTTCAAAGGACAA-3′ | TGG |
| 2 | 5′-TGTCTTGTTCAAAGGACAAT-3′ | GGG |
| 3 | 5′-GGTGTCCATAGTACCCGGAT-3′ | GGG |
| 4 | 5′-CGGTGTCCATAGTACCCGGA-3′ | TGG |
| 5 | 5′-GGCTCGGTGTCCATAGTACC-3′ | CGG |

II. constructing a CRISPR-Cas9 recombinant lentivirus vector for knocking out the RSPO2 gene with the specificity

1. constructing sgRNA oligo, which specifically comprises steps of:

(1) adding a CACC (complementary sequence of BsmBI cutting site cohesive ends) and a G (to ensure the effective U6 promoter) on a 5′ end of a corresponding DNA sequence to obtain a forward oligo based on the selected sgRNA;

(2) obtaining a complementary strand of a corresponding DNA based on the selected sgRNA; adding an AAAC (complementary sequence of BsmBI cutting site sticky ends) on the 5′ end of the corresponding DNA sequence and adding a C on a 3′ end to obtain a reverse oligo;

(3) oligo formats are:

forward: 5′-CACC-G-(20N)-3′

reverse: 5′-AAAC-(20N complementary sequence)-C-3′; and

(4) synthesizing the forward oligo and the reverse oligo respectively, as shown in Table 2.

TABLE 2
oligo sequences of the sgRNA for specifically
knocking out human RSPO2

| oligo | oligo sequence |
|---|---|
| forward oligo (1) | 5′-CACCGTTGTCTTGTTCAAAGGACAA-3′ |
| reverse oligo (1) | 5′-AAACTTGTCCTTTGAACAAGACAAC-3′ |
| forward oligo (2) | 5′-CAACGTGTCTTGTTCAAAGGACAAT-3′ |
| reverse oligo (2) | 5′-AAACATTGTCCTTTGAACAAGACAC-3′ |
| forward oligo (3) | 5′-CACCGGGTGTCCATAGTACCCGGAT-3′ |
| reverse oligo (3) | 5′-AAACATCCGGGTACTATGGACACCC-3′ |
| forward oligo (4) | 5′-CACCGCGGTGTCCATAGTACCCGGA-3′ |
| reverse oligo (4) | 5-AAACTCCGGGTACTATGGACACCGC-3′ |
| forward oligo (5) | 5′-CACCGGGCTCGGTGTCCATAGTACC-3′ |
| reverse oligo (5) | 5′-AAACGGTACTATGGACACCGAGCCC-3′ |

2. linearizing and recovering the vector

The lentiviral vector adopts a lentiCRISPR V2 (Feng Zhang, Nature Methods, 2014); the lentiviral vector contains Cas9 and sgRAN framework, as well as the U6 promoter to control sgRNA expression, so as to insert a sgRNA fragment containing a BsmBI cohesive end after being digested with BsmBI;

wherein the lentiCRISPR is adopted as a BmsBI digestion vector; a DNA purification kit is adopted to purify and recover a digestion product.

3. phosphorylating, annealing and connecting the oligos to the lentiCRISPR, which specifically comprises steps of:

(1) annealing a phosphorylated product of the forward oligo and the reverse oligo, so as to generate the fragment with the BsmBI cohesive end; and

(2) ligating the fragments to the lentiCRISPR to form the CRISPR-Cas9 recombinant lentivirus vector.

4. transforming and sequencing

An Escherichia coli DH5a is transformed, wherein a positive clone is screened and a sequence is identified.

5. transfecting 293FT cells, amplifying the RSPO2 gene with PCR, and identifying with T7EI digestion.

III. processing the CRISPR-Cas9 for knocking out the sgRNA of the human RSPO2 gene with lentiviral packaging

The lentiviral packaging system is a four-plasmid system (Shanghai Genepharma Co., Ltd) which comprises a shuttle vector, PG-p1-VSVG, PG-P2-REV and PG-P3-RRE; wherein the shuttle vector is able to express the target gene; PG-p1-VSVG, PG-P2-REV and PG-P3-RRE contain necessary elements of the lentiviral packaging. The 293FT cells are transfected by the above lentiviral vector and lentiviral-packaged system, the transfected cells are collected, centrifuged and filtered, and the lentivirus titer kit is used to detect lentivirus titer

Benefits of the present invention are as follow. The present invention disclosed the method for knocking out human RSPO2 gene with the CRISPR-Cas9 specificity, which is applied in research of liver fibrosis. The CRISPR-Cas9 specificity is able to inhibit the human RSPO2 gene expression, and is able to repress the competence of the Wnt signal pathway when transfected hepatic stellate cell, which significantly down-regulates the markers α-SMA and Collagen I of liver fibrosis. The present invention adopts the CRISPR-Cas9 for the RSPO2 gene target to effectively suppress the hepatic stellate cell activation and provide an effective way to cure liver fibrosis.

Meanwhile, the present invention discloses a method for effectively and specifically knocking out the RSPO2 gene by using CRISPR-Cas9, which effectively solves the problems of using the RSPO antibody in treating fibrosis: (1) directly knocking out the RSPO2 gene can achieve long-term inhibition effect on target gene; (2) lentivirus or adenovirus vector is used for stable intracellular transcription of sgRNA, play a long-term inhibition effect on target gene expression. (3) simultaneous knockout of multiple coding sequences of RSPO2 is achieved, and even simultaneous knockout of multiple target genes can be achieved; and (4) extracellular and intracellular targets are simultaneously targeted.

### BRIEF DESCRIPTION OF THE DRAWINGS

FIG. 1 illustrates a principal of knocking out a human RSPO2 gene with CRISPR-Cas9 specificity.

FIG. 2 illustrates sgRNA, wherein GGG is PAM and a sequence below is sgRNA.

FIG. 3 is a test result of transforming an Escherichia coli DH5a by lentiviral vector plasmids designed for a RSPO2 gene at No. 1, 2, 3, 4 and 5 targets.

FIG. 4 is a test result of transfecting 293FT cells with the lentiviral vector plasmids designed for the RSPO2 gene at the No. 1, 2, 3, 4 and 5 targets, collecting the transfected cells after 48 hours, amplifying the RSPO2 gene by PCR and verifying with T7EI.

FIG. 5 illustrates QPCR (quantitative polymerase chain reaction) verification of knocking out the RSPO2 gene of hepatic stellate cells with CRISPR-Cas9 specificity, indicating a mRNA level of the RSPO2 is lowered.

FIG. 6 illustrates Western Blot verification of knocking out the RSPO2 gene of hepatic stellate cells with CRISPR-Cas9, indicating a RSPO2 protein level is lowered.

FIG. 7 illustrates immunofluorescence verification of knocking out the No. 1 target of the RSPO2 gene of hepatic stellate cells with CRISPR-Cas9, indicating the fibrosis of the hepatic stellate cell is inhibited.

FIG. 8 illustrates MTT proliferation test verification of knocking out the No. 1 target of the RSPO2 gene of hepatic stellate cells with CRISPR-Cas9, indicating proliferation of the hepatic stellate cell is inhibited.

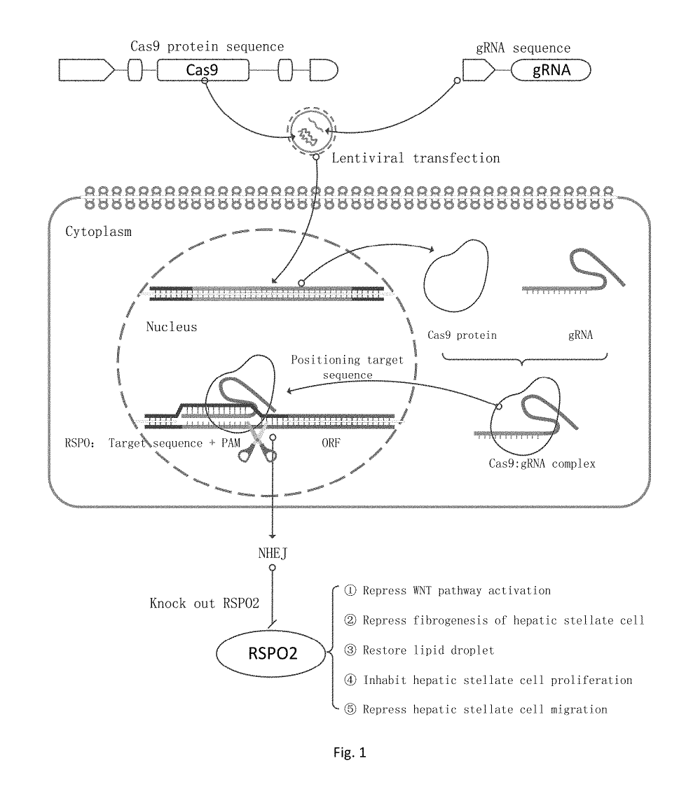

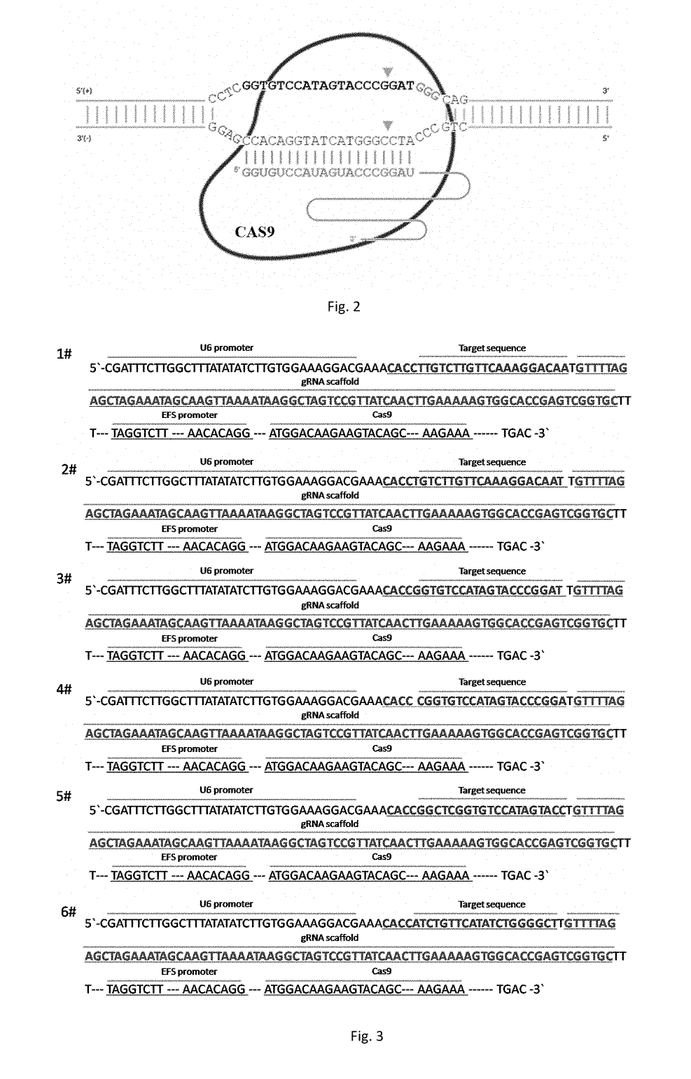

### DETAILED DESCRIPTION OF THE PREFERRED EMBODIMENT

Referring to the drawings, according to preferred embodiments the present invention is further illustrated. The embodiments are for explaining the present invention and not a limitation to the present invention.

The various sgRNA are able to be used in combination of two of more sgRNA. By combination, the CRISPR-Cas9 is able to target multiple targets and knock out the human RSPO2 gene effectively.

The following embodiments are not independent but consecutive process. The molecular biology technologies involved in the embodiments include the cell culture, vector construction, cell transfection, clone, gene sequencing, Western blot test, PCR amplification and test and immunofluorescence. Except explained otherwise, the technologies adopted are regular technologies which are understandable by a skilled technician in the field and the instruments, reagents, plasmid, cell strain and etc. are able to be approached by a skilled technician in the field through public channel.

### Embodiment 1: Designing a sgRNA Sequence

Based on the experiences, the sgRNA sequence is designed to satisfy the follow conditions: (1) a length of the sgRNA is 20 nucleotide sequences; (2) a sgRNA target on the RSPO2 gene locates in an exon thereof, which easily leads to deletion of gene fragments or frameshift mutations, so as to complete gene inactivation; (3) preferably, the sgRNA target on the RSPO2 gene locates in a functional domain thereof, so as to complete gene inactivation more easily; (4) Blast in a NCBI database is used to identify unique sgRNA target sequence, reducing potential off-target sites; (5) 5′-NGG is selected for PAM of a target sequence; (6) preferably, a sgRNA target sequence is started at G to ensure an effective U6 promoter of a vector; and (7) a format of the sgRNA target sequence is as follows:

forward oligo: 5′-G-(19N)-NGG-3′

reverse oligo: 5′-CCN-(19N)-C-3′ (19N denote 19 nucleotide sequences of the target)

or

forward oligo: 5′-(20N)-NGG-3′

reverse oligo: 5′-CCN-(20N)-C-3′

The sgRNA sequences of the targeted RSPO2 gene are designed based on the conditions, from which 20 sgRNA sequences of the targeted RSPO2 gene are selected as examples to illustrate the present invention. The 20 sgRNA sequences are listed in the sequence list as SEQ ID NO. 2, 4, 6, 8, 10, 12, 14, 16, 18, 20, 22, 24, 26, 28, 30, 32, 34, to 36, 38, 40; the corresponding DNA target sequences are listed in the sequence list with singular numbers SEQ ID NO.1, 3, 5, 7, 9, 11, 13, 15, 17, 19, 21, 23, 25, 27, 29, 31, 33, 35, 37, 39 (wherein 1-20 is the target sequence, the last three are PAM sequences).

### Embodiment 2: Selecting the sgRNA Sequence

Paralogy analysis is provided between candidate sgRNA sequences and a genome database by adopting Blast (www.ncbi.nlm.nig.gov/Blast) to ensure the uniqueness of the sgRNA which is not paralogous to the gene sequences other than the human RSPO2 genes. The sgRNA sequences for effectively knocking out human RSPO2 genes are selected based on the following rules: (1) the sgRNA target locates in DHSs (DNase hypersensitive sites); (2) the sgRNA target is not to close to a start cordon (ATG); (3) an off-target rate is low.

Five sgRNA sequences corresponding to different targets of the targeted human RSPO2 genes satisfy the rules and are selected from 20 sgRNA sequences of the targeted human RSPO2 genes (corresponding to SEQ ID NO.2, 4, 6, 8, 10 in the sequence list), and the other fifteen cannot satisfy the rules. The sgRNA target sequences and the corresponding PAM sequences are listed in Table 1 (corresponding to SEQ ID NO.1, 3, 5, 7, 9 in the sequence list).

### Embodiment 3: Synthesizing sgRNA Oligo

Adding BsmBI cutting site onto a 5′ end of the sgRNA sequences of the targeted human RSPO2 genes comprises steps of:

(1) adding a CACC (complementary sequence of BsmBI cutting site cohesive ends) and a G (to ensure the effective U6 promoter) on a 5′ end of a corresponding DNA sequence to obtain a forward oligo based on the selected sgRNA; and (2) obtaining a complementary strand of a corresponding DNA based on the selected sgRNA; adding a to AAAC (complementary sequence of BsmBI cutting site sticky ends) on the 5′ end of the corresponding DNA sequence and adding a C on a 3′ end to obtain a reverse oligo; and (3) synthesizing the forward oligo and the reverse oligo by chemical synthesis method to obtain the oligos as shown in the table 2.

### Embodiment 4: Constructing a Lentiviral Vector

Annealing and ligating the synthesized five pairs of oligo single-chain fragments (referring to Table. 2) to the lentiviral vector which respectively transcribes the sgRNA of the targeted RSPO2 with specificity comprises steps of:

1. linearizing and recovering the vector

The lentiviral vector adopts a lentiCRISPR V2 (Feng Zhang, Nature Methods, 2014); the lentiviral vector contains Cas9 and sgRAN framework, as well as the U6 promoter to control sgRNA expression, and a EFS-NS promoter to control Cas9 expression, so as to insert a sgRNA fragment containing a BsmBI cohesive end after being digested with BsmBI.

1) digesting the lentiCRISPR plasmid by BmsBI, wherein the digesting system is as follow:

| | |
|---|---|
| lentiCRISPR | 5 μl (400 ng/μl) |
| 10x Buffer | 2 μl |
| BmsB I | 1 μl |
| Dnase/Rnase-Free H2O | 12 μl |

2) incubating for 3 to 4 hours at 37° C.; and

3) recovering the digesting products by a DNA purification kit.

2. phosphorylating the oligo, wherein phosphorylating the synthesized oligos by adopting T4 polyphosphate kinase (Takara);

3. annealing the oligos, wherein

1) establishing the following annealing reaction system (room temperature) in the sterile centrifuge tubes;

| | |
|---|---|
| oligos sense strand | 10 μl |
| oligos antisense strand | 10 μl |
| 5x DNA annealing buffer | 10 μl |
| Dnase/Rnase-Free H2O | 20 μl |

2) incubating for 4 minutes at 95° C.; incubating for 10 minutes at 70° C.;

3) taking out of the centrifuge tubes; placing at the room temperature for 5-10 minutes; cooling down to the room temperature; and

4) centrifuging for a short time; blending.

4. ligating to the vector, wherein

1) ligating the annealing products to the lentiCRISPR vector with a ligation system as follow:

| | |
|---|---|
| products of the oligos annealing | 4 μl |
| products of lentiCRISPR enzyme cutting recovering | 1 μl |
| T4 ligase | 5 μl |
| Dnase/Rnase-Free H2O | 10 μl |

2) incubating for 1 hour at 1° C. to obtain lentivial vector plasmids (1) lenti_sgRNA_RSPO2_1, (2) lenti_sgRNA_RSPO2_2, (3) lenti_sgRNA_RSPO2_3, (4) lenti_sgRNA_RSPO2_4, and (5) lenti_sgRNA_RSPO2_5.

5. transforming Escherichia coli DH5a, wherein

1) adding the ligation products (1) lenti_sgRNA_RSPO2_1, (2) lenti_sgRNA_RSPO2_2, (3) lenti_sgRNA_RSPO2_3, (4) lenti_sgRNA_RSPO2_4, and (5) lenti_sgRNA_RSPO2_5 of 10 μl respectively into the 100 μl DH5a competent cell; blowing even; settling in the ice for 20 minutes; water bathing for 90 s at 42° C.; rapidly ice bathing for 3 minutes; adding 500 μl LB liquid culture media; putting in the shaker 180 rpm for 1 hour at 37° C.;

2) coating the bacteria liquid of 100 μl on the LB solid culture medium (containing 1/1000 ampicillin); incubating overnight at 37° C.;

6. screening the positive clone and sequencing identification, comprising steps of:

1) selecting colony PCR and initially identifying the positive clone; wherein:

the primer sequence is as follow:

upstream primer:
5′-GAGGGCCTATTCCCATGATTCCTTCATAT-3′;
downstream primer:
5′-CATAGCGTAAAAGGAGCAAC-3′;

PCR system:

| | |
|---|---|
| 2x PCR buffer | 25 ul |
| upstream primer (25 pmol/L) | 1 ul |
| downstream primer (25 pmol/L) | 1 ul |
| bacteria liquid | 2 ul |
| deionized water | 21 ul |

amplification conditions: 10 minutes at 94° C., one cycle; 30 seconds at 94° C., 30 seconds at 55° C., 30 seconds at 72° C., 30 cycles; 6 minutes at 72° C., one cycle;

2) screening the positive clone for further sequencing analysis; wherein the sequencing result (referring to the FIG. 3) shows a successful lentiviral vector construction.

### Embodiment 5: Verifying Endogenous Activity of sgRNA Targets

293FT cells are transfected with the above lentiviral vector, RSPO2 gene is amplified by PCR, and endogenous activity of sgRNA target is identified by T7EI digestion.

1. transfecting the 293FT cells with the constructed lentiviral vector plasmid

1) seeding 293FT cells in a 96-well plate at 2×10⁴ cells/well and culturing in a high glucose DMEM medium containing 10% fetal bovine serum at 37° C. in a 5% CO₂ incubator;

2) replacing the cell culture medium by a serum-free medium 2 h before infection;

3) transfecting the lentiviral vector into six groups when the degree of cell fusion reaches 70%; wherein the six groups are (1) lenti_sgRNA_RSPO2_1, (2) lenti_sgRNA_RSPO2_2, (3) lenti_sgRNA_RSPO2_3, (4) lenti_sgRNA_RSPO2_4, (5) lenti_sgRNA_RSPO2_5, and (6) negative control group; and a reaction system is as follows:

| | |
|---|---|
| lentiviral vector plasmid | 0.1 μg/well |
| lipofectamine 2000 | 0.6 μl/well |

4) harvesting cells 48 hours after transfection;

2. sorting positive cells using a flow cytometry;

3. extracting DNA from positive cells and amplifying the RSPO2 gene by PCR

the primer sequence is:

upstream primer:
5′-GTTTCCTCAGGGCATTGCTT-3′
downstream primer:
5′-TGCATTATTTCCCTGGCTGA-3′

amplification conditions: 95° C. for 3 minutes, 1 cycle; 94° C. for 30 seconds, 55° C. for 30 seconds, 30 cycles; 72° C. for 6 minutes, 1 cycle.

4. identifying with T7EI digestion

digesting recycled PCR products with T7 Endonuclease I digestion identification, wherein a digesting system is:

| | |
|---|---|
| T7EI | 1 μl |
| buffer | 2 μl |
| PCR product | 10 μl |
| deionized water | 7 μl |

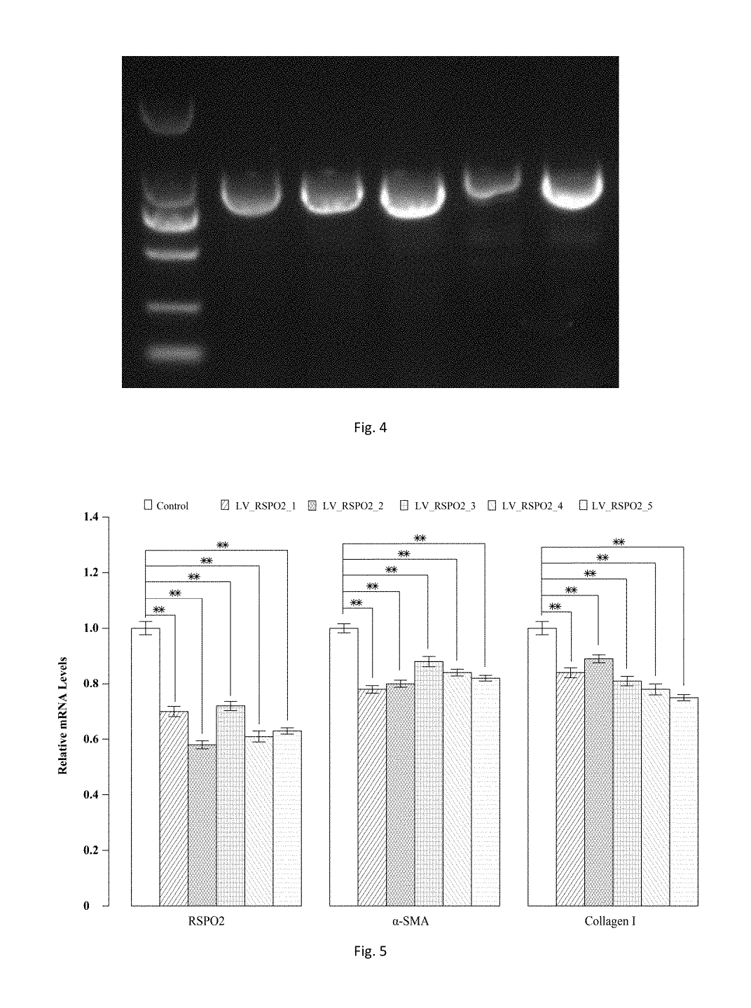

using 37° C. water bath for 45 minutes, then detecting 10 μl digested products by agarose gel electrophoresis. The results indicate (referring to the FIG. 4) that there are varying degrees of mutation at each target site for the RSPO2 gene.

### Embodiment 6: Validating the Lentiviral Vector

1. seeding 293FT cells in a 96-well plate at 2×10⁴ cells/well and culturing in a high glucose DMEM medium containing 10% fetal bovine serum at 37° C. in a 5% CO₂ incubator;

2. replacing the cell culture medium by a serum-free medium 2 h before infection;

3. transfecting the lentiviral vector into six groups when the degree of cell fusion reaches 70%; wherein the six groups are (1) lenti_sgRNA_RSPO2_1, (2) lenti_sgRNA_RSPO2_2, (3) lenti_sgRNA_RSPO2_3, (4) lenti_sgRNA_RSPO2_4, (5) lenti_sgRNA_RSPO2_5, and (6) negative control group; and a reaction system is as follows:

| | |
|---|---|
| lentiviral vector plasmid | 0.1 μg/well |
| lipofectamine 2000 | 0.6 μl/well |

4. harvesting cells 48 hours after transfection;

5. detecting fluorescence intensity of the sample by a microplate reader with an excitation of 485 nm and an emission of 533 nm; and

6. calculating fluorescence intensity:

fluorescence intensity=(transfection group fluorescence intensity−non-transfection group fluorescence intensity)/non-transfection group fluorescence intensity

The results show that lentiviral vectors (lenti_sgRNA_RSPO2_1, lenti_sgRNA_RSPO2_2, lenti_sgRNA_RSPO2_3, lenti_sgRNA_RSPO2_4, and lenti_sgRNA_RSPO2_5) of CRISPR-Cas9 system knocking out of the RSPO2 with the specificity can effectively inhibit RSPO2 gene expression.

### Embodiment 7: Packaging Lentiviral

The lentiviral packaging system is a four-plasmid system (Shanghai Genepharma Co., Ltd) which comprises a shuttle vector, PG-p1-VSVG, PG-P2-REV and PG-P3-RRE; wherein the shuttle vector is able to express the target gene; PG-p1-VSVG, PG-P2-REV and PG-P3-RRE contain necessary elements of the lentiviral packaging.

1. cell strain

digesting the well developed 293T cell with 0.25% pancreatin; inoculating the 293T cell in a 10 cm cell culture dish (about 2-2.5×10⁶ cells in each cell culture dish); cultivating the cells in the CO₂ incubator at 37° C.;

2. lentiviral packaging

preparing the lentiviral vector (lenti_sgRNA_RSPO2_1, lenti_sgRNA_RSPO2_2, lenti_sgRNA_RSPO2_3, lenti_sgRNA_RSPO2_4, and lenti_sgRNA_RSPO2_5) according to the following method:

1) the reaction system of the lentiviral packaging is as follow:

| | |
|---|---|
| expression vector | 20 μg |
| PG-p1-VSVG vector | 10 μg |
| PG-P2-REV vector | 10 μg |
| PG-P3-RRE vector | 10 μg |
| serum-free Opti-MEM | 750 μl |
| RNAi-Mate | 300 μl |

wherein the total regulated volume is 2.5 ml; the lentiviral packaging system is incubated for 5 minutes at a room temperature;

2) mixing 100 μl Lipofectamine2000 reagent with 2.4 ml Opti-MEM in another tube; incubating for 5 minutes at room temperature; mixing the diluted DNA and diluted Lipofectamine2000; reverse mixing for 5 minutes; incubating for 20 minutes at room temperature; and

3) transforming the mixture of the DNA and the Lipofectamine2000 to the 293T cell culture media and blending; cultivating for 4-6 hours and replacing the culture media with DMEM (+10% FBS) culture media; cultivating in CO₂ incubator for 48 hours;

3. collecting and concentrating the lentivirus;

1) collecting the 293FT cell supernatant after transfecting for 48-72 hours (transfecting start at 0 hour);

2) centrifuging for 4000 g at 4° C. and removing the cell debris;

3) filtering the supernatant with 0.45 filter in 40 ml ultra centrifugal;

4) adding the crude extract of the lentivirus sample into the filtering cup (19 ml at the most); inserting the filtering cup into the filtrate collecting tube;

5) centrifuging in 4000×g until the concentration volume reaches the requirement, which need 10-15 minutes;

6) taking out the filtering cup after centrifuging; separating the filtering cup with the collected liquid;

7) centrifuging under 1000 g for 2 minutes;

8) obtaining the lentivirus LV_RSPO2_1, LV_RSPO2_2, LV-RSPO2_3, LV_RSPO2_4 and LV-RSPO2_5 from the lentivirus concentration in the sample collecting cup; and

9) removing the lentivirus concentration to the lentivirus tubes after separation; storing for a long term at −80° C.

4. titrating the lentivirus;

titrating the lentivirus by adopting the quickTiter quicktiter lentivirus titer kit, comprising the following steps:

1) preparing and blending the reagents according to instructions;

2) preparing two parallel holes for each lentivirus sample, standard lentivirus liquid, blank and the control;

3) adding 100 μl deactivated lentivirus sample and standard P24 antigen into the antibody coating plates;

4) sealing the 96-well plate with the sealing film and incubating for 4 hours at 37° C.;

5) removing the sealing film, discarding the liquid in the 96-well plate and washing the plate with 250 μl 1× scrubbing solution for three times; drying the plates;

6) adding 100 μl diluted FITC marked single clone antibody for p24 in each well;

7) sealing the 96-well plate with the sealing film; placing the 96-well plate in the shaker; incubating for 1 hour at room temperature;

8) removing the sealing film, discarding the liquid in the 96-well plate and washing the plate for three times;

9) adding 1000 diluted HRP marked single clone antibody for FITC in each well; sealing the 96-well plate with the sealing film; placing the 96-well plate in the shaker; incubating for 1 hour at room temperature;

10) removing the sealing film discarding the liquid in the 96-well plate, washing the plate for three times and rapidly go to the next step;

11) balancing the substrate solution to the room temperature; adding 1000 substrate solution in each well including the blank plate; placing the 96-well plate on the shaker; incubating for 20-30 minutes at room temperature; and

12) stopping the reaction by adding 1000 stopping solution in each well; testing the absorbance of each well at 450 nm wave length by microplate reader;

calculating the amount of the lentivirus p24 protein; wherein each lentivirus particle (LP) contains around 2000 p24 molecular; obtaining the lentivirus titer according to the formula:

1 ng p24=1.25×10⁷LP;

TABLE 3
various lentivirus titer

| lentivirus | titer |
|---|---|
| LV_RSPO2_1 | 3.13 × 10⁶ LP |
| LV_RSPO2_2 | 3.47 × 10⁶ LP |
| LV_RSPO2_3 | 4.09 × 10⁶ LP |
| LV_RSPO2_4 | 3.74 × 10⁶ LP |
| LV_RSPO2_5 | 2.03 × 10⁶ LP |

### Embodiment 8: Transfecting the Human Hepatic Stellate Cell Strain

1) cultivating the human hepatic stellate cell LX2; inoculating the cell suspension in the 12-well plate; cultivating in the 5% CO₂ incubator at 37° C.;

2) dividing the cell into groups when the cell confluence reaches 30% to 40%; wherein the groups are as follow: (1) negative control group: for negative controlling lentivirus particle transfection cell; (2) RSPO2_1 experimental group; transfecting the cell with the lentiviral vectors LV_RSPO2_1; (3) RSPO2_2 experimental group; transfecting the cell with the lentiviral vectors LV_RSPO2_2; (4) RSPO2_3 experimental group; transfecting the cell with the lentiviral vectors LV_RSPO2_3; (5) RSPO2_4 experimental group; transfecting the cell with the lentiviral vectors LV_RSPO2_4; (6) RSPO2_5 experimental group; transfecting the cell with the lentiviral vectors LV_RSPO2_5;

3) taking out the lentivirus stored at 4° C.; centrifuging for 20 seconds with stationary centrifugal; diluting the lentivirus with MOI 0.2 in the culture media; minimizing the volume of the culture media containing the lentivirus as long as possible to obtain a preferred transfection efficiency;

4) transfecting the lentivirus when the cell confluence reaches 70%;

a) absorbing an accurate volume of the lentivirus liquid with a pipet; adding the lentivirus liquid in the prepared culture media;

b) absorbing the culture media in the original cell culture media (if the cells grow well with a preferred density, no need to replace the culture media);

c) adding the calculated lentivirus liquid in the target cell and the control cell;

d) incubating in the CO₂ incubator (37° C., 5% CO₂) overnight after blending;

5) observing the cell after 12 hours; if no obvious cytotoxicity appears, continue the cultivation for 48 hours before replacing the culture media; if appears obvious cytotoxicity, replace the culture media immediately; and

6) observing the expression of the lentivirus reporter's green fluorescent protein (GFP) 4 to 5 days after the infection; if the infection efficiency is below 50%, re-infects the cell; if the infection efficiency is over 50%, collects the cell for further test.

### Embodiment 9: Providing QPCR Test

Transfecting the human hepatic stellate cell LX2 with the constructed lentivirus as illustrated in the embodiment 8; and processing the mRNA level of the RSPO2 and the marker (α-SMA, Collagen-I) of liver fibrosis with QPCR test;

1) PCR primers are illustrated as follow:

TABLE 4
QPCR primer

| gene | forward | reverse |
|---|---|---|
| RSPO2 | 5′-GTTTCCTCAGGG | 5′-TGCATTATTTCCC |
| | CATTGCTT-3′ | TGGCTGA-3′ |
| α-SMA | 5′-GCATCTGGGTGA | 5′-GCAATGCCTCTGA |
| | AAAGTGGT-3′ | TTTCCAT-3′ |
| Collagen-I | 5′-CCAAATCTGTCT | 5′-TCAAAAACGAAGG |
| | CCCCAGAA-3′ | GGAGATG-3′ |
| β-actin | 5′-GAAGCTGTGCTA | 5′-CAATAGTGATGAC |
| | TGTTGCTCTA-3′ | CTGGCCGT-3′ |

2) extracting the RNA by Trizol; storing RNA at −80° C.;

3) determining the absorbance at 260 nm and 280 nm wavelength by the ultraviolet Spectrometer; calculating the concentration of the extracted RNA;

4) reverse transcribing and synthesizing cDNA by the reverse transcribing kit; the reaction system is as follow:

| | |
|---|---|
| 2x RT buffer | 10 μl; |
| 6N random primer(100 pmol/μl) | 1 μl |
| RT-mix | 1 μl |
| Template (RNA) | 5 μl |
| DEPC water | 3 μl |

10 minutes at 20° C., 50 minutes at 42° C., 5 minutes at 85° C.; storing at −20° C.

5) PCR reaction system is as follow:

| | |
|---|---|
| SYBR green I | 0.5 μl |
| 2x PCR buffer | 25 μl |
| Upstream primer(25 pmol/L) | 1 μl |
| Downstream primer(25 pmol/L) | 1 μl |
| Sample cDNA | 2 μl |
| DEPC water | 20.5 μl |

reacting on the ABI 7500 PCR instrument;

6) PCR conditions: 4 minutes at 94° C., one cycle; 20 seconds at 94° C., 30 seconds at 60° C., 30 seconds at 72° C., 35 cycles; extending for 5 minutes at 72° C.; and

7) analyzing the data with SDS software; analyzing the result by comparing the Ct value; standardizing the expression value of the target gene by β-actin.

QPCR test shows that the mRNA level of the RSPO2 of the human hepatic stellate cell and the marker of liver fibrosis α-SMA and Collagen-I (referring to the FIG. 5) is down-regulated significantly in the LV_RSPO2_1, LV_RSPO2_2, LV_RSPO2_3, LV_RSPO2_4, LVRSPO2_5 groups comparing to the control group. The CRISPR-Cas9 system designed by the present invention inhabits the RSPO2 target gene expression and represses the activation of the hepatic stellate cell.

### Embodiment 10: Providing Western Blot Testing

Transfecting the human hepatic stellate cell with the constructed lentivirus as illustrated in the embodiment 8; testing the expression of the RSPO2 protein in the hepatic stellate cell LX2 by the Western blot, which comprises steps of:

1) extracting the protein of hepatic stellate cell by RIPA lysis buffer;

2) testing the absorbance of the various wells at 562 nm wavelength by the microplate reader; calculating the protein concentration according to the standard curve;

3) separating by polyacrylamide gel electrophoresis, transmembraning and sealing with 5% skimmed milk powder; adding RSPO2 antibody (1:1000), α-SMA antibody (1:300) and Collagen-41:1000) respectively; incubating overnight at 4° C.;

4) adding secondary antibodies (1:2000) after washing; incubating for two hours at room temperature before ECL (electrogenerated chemiluminescence) testing; and

5) taking β-actin as the internal reference to analyze the grey scale of the various stripes by the gel image system (Bio-Rad Laboratories AB);

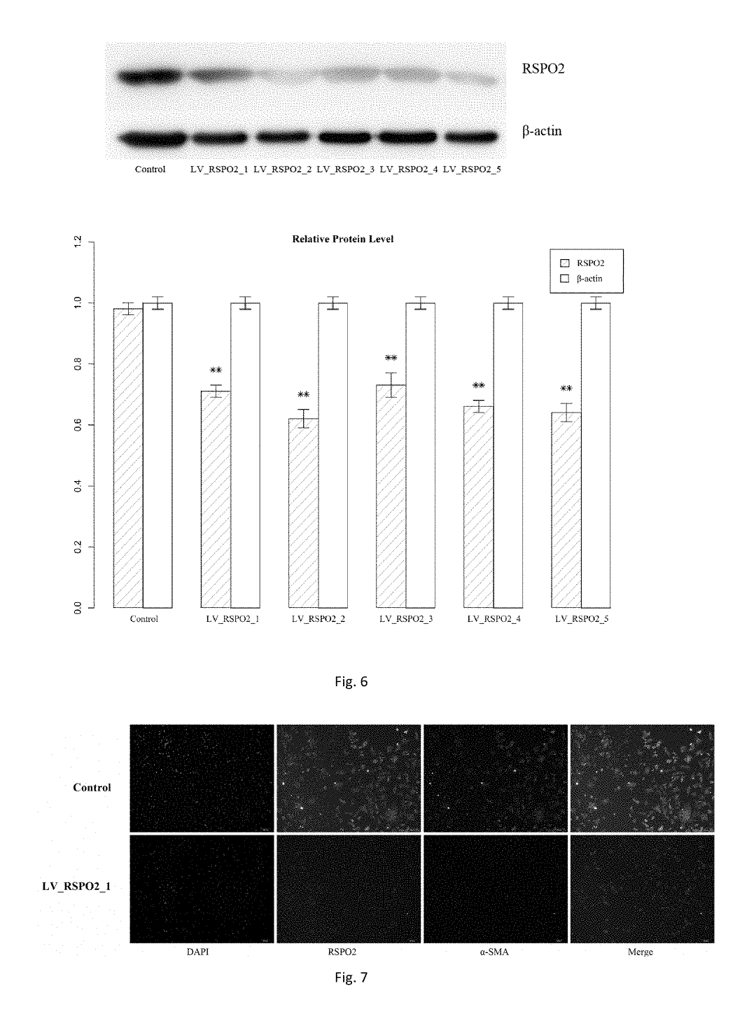

Western blot test shows that the expression of the RSPO2 protein of the hepatic stellate cell LX2 (referring to the FIG. 6) is down-regulated significantly in the LV_RSPO2_1, LV_RSPO2_2, LV_RSPO2_3, LV_RSPO2_4, LVRSPO2_5 groups comparing to the control group. The CRISPR-Cas9 system designed by the present invention inhibits the RSPO2 target gene expression and effectively represses the activation of the hepatic stellate cell.

### Embodiment 11: Providing Immunofluorescence Test

Taking the LV_RSPO2_1 as an example, transfecting the human hepatic stellate cell with the constructed lentivirus as illustrated in the embodiment 8; testing the expression of the RSPO2 protein in the hepatic stellate cell LX2 and the marker of liver fibrosis α-SMA by immunofluorescence testing; comprising the following steps:

1) discarding the culture media for the transfected hepatic stellate cell by the lentivirus LV_RSPO2_1; washing the cell with the incubated PBS for 10 minutes for two times respectively; fixing the cells for 15 minutes by 4% POM (Polyoxymethylene) at the room temperature;

2) washing the cells for 10 minutes for two times respectively by PBS; permeating the membrane with 0.1% Triton X-100 at 4° C. for 15 minutes;

3) washing the cells for 10 minutes for two times respectively by PBS; sealing the cells with 4% BSA for 30 minutes at room temperature;

4) diluting the various primary antibodies (RSPO2 and α-SMA) by 1:100; incubating overnight in the refrigerator at 4° C.;

5) washing the cells for 10 minutes for three times respectively by PBS; diluting the various secondary antibodies by 1:100; incubating for one hour at 37° C.; and

6) washing the cells for 10 minutes for three times respectively with PBS; staining the nucleus with DAPI (4′,6-diamidino-2-phenylindole) and shooting with fluorescence microscope;

The immunofluorescence test shows that the expression of the RSPO2 protein and the α-SMA protein of the hepatic stellate cell (referring to the FIG. 7) are down-regulated significantly comparing to the control group. The CRISPR-Cas9 system designed by the present invention inhibits the RSPO2 target gene expression and represses the activation of the hepatic stellate cell.

### Embodiment 12: Providing MTT Proliferation Test

Taking the LV_RSPO2_1 as an example, transfecting the hepatic stellate cell LX2 with the constructed lentivirus as illustrated in the embodiment 8; testing the proliferation of the hepatic stellate cell by MTT; comprising the following steps:

1) inoculating the hepatic stellate cell in the 96-well culture plate; wherein the cell density of the each well is 4×10³;

2) transfecting the LV_RSPO2_1 lentiviral vector in the control group as illustrated in the embodiment 4;

3) transfecting for 24 hours, 48 hours and 72 hours; adding 10 μl MTT liquid in each orifice;

4) incubating for 4 hours at 37° C.; adding 100 μl DMSO in each well; blending even; and

5) testing the absorbance at 570 nm wavelength by the microplate reader; calculating the cell survival rate.

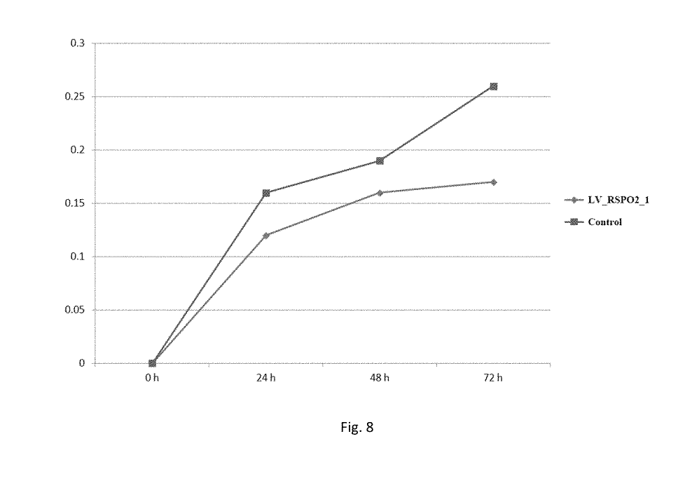

The MTT test shows that the proliferation of the hepatic stellate cell is down-regulated significantly comparing to the control group (referring to the FIG. 8). The CRISPR-Cas9 system designed by the present invention inhibits the RSPO2 target gene expression and effectively represses the proliferation of the hepatic stellate cell.

It can be seen from the above embodiments that the CRISPR-Cas9 system of the present invention can knock out the human RSPO2 gene with high knockout efficiency.

The preferred embodiments of the present invention are described in detail above. However, the present invention is not limited to the specific details of the above embodiments. Various simple modifications may be made to the technical solutions of the present invention within the scope of the technical concept of the present invention. All belong to the protection scope of the present invention.

In addition, it should be noted that each of the specific technical features described in the foregoing specific embodiments may be combined in any suitable manner without contradiction. In order to avoid unnecessary repetition, the present invention is applicable to various possible ways of combination and will not be described to separately. In addition, any combination of various embodiments of the present invention may also be adopted as long as it does not violate the spirit of the present invention, and should also be regarded as the disclosure of the present invention.

What is claimed is:

1. A method for knocking out a human RSPO2 (R-spondin 2) gene targeted with CRISPR-Cas9 (clustered regularly interspaced short palindromic repeats associated) specificity, comprising steps of:
1) designing the sgRNA of the human RSPO2 gene targeted; and
2) constructing a CRISPR-Cas9 recombinant lentivirus vector for knocking out the RSPO2 gene.

2. The method, as recited in claim 1, wherein the step 1) specifically comprises steps of:
1-1) designing the sgRNA of the targeted human RSPO2 gene based on first preset conditions; and
1-2) selecting the sgRNA of the targeted human RSPO2 gene based on second preset conditions.

3. The method, as recited in claim 2, wherein the first preset conditions comprise:
(1) a length of the sgRNA is 20 nucleotide sequences;
(2) a sgRNA target on the RSPO2 gene locates in an exon thereof;
(3) the sgRNA target on the RSPO2 gene locates in a functional domain thereof;
(4) 5′-NGG is selected for PAM of a target sequence;
(5) a sgRNA target sequence is started at G to ensure an effective U6 promoter of a vector; and
(6) a format of the sgRNA target sequence is as follows:
5′-G-(19N)-NGG-3′ only when the sgRNA target sequence starts at G; or
5′-(20N)-NGG-3′;
wherein, 19N or 20N refers to 19 or 20 nucleotide sequences of the sgRNA target.

4. The method, as recited in claim 2, wherein the second preset conditions comprise:
(1) in a NCBI (National Center for Biotechnology Information) database, a BLAST (Basic Local Alignment Search Tool) is adopted to ensure a uniqueness of the sgRNA target sequence which is not paralogous with gene sequences other than the human RSPO2 gene;
(2) the sgRNA target locates in DHSs (DNase I hypersensitive sites);
(3) there is a certain distance between the sgRNA target and a start cordon ATG; and
(4) an off-target rate is low.

5. The method, as recited in claim 1, wherein the step 2) specifically comprises steps of:
2-1) constructing sgRNA oligos;
2-2) linearizing and recovering a lentiviral vector; wherein the lentiviral vector adopts lentiCRISPR; the lentiviral vector contains Cas9 and a sgRAN framework, as well as a U6 promoter to control sgRNA expression, so as to insert a sgRNA fragment containing a BsmBI cohesive end after being digested with BsmBI;
wherein the lentiCRISPR is adopted as a BmsBI digestion vector; a DNA purification kit is adopted to purify and recover a digestion product.
2-3) phosphorylating, annealing and ligating the sgRNA oligos to the lentiCRISPR;
2-4) transforming Escherichia coli DH5a, screening a positive clone and sequencing; and
2-5) transfecting 293FT cells, amplifying the RSPO2 gene with PCR, and identifying with T7EI digestion.

6. The method, as recited in claim 5, wherein the step 2-1) specifically comprises steps of:
2-1-1) adding a CACC and a G on a 5′ end of a corresponding DNA sequence of the selected sgRNA to obtain a forward oligo of 5′-CACC-G-(20N)-3′;
2-1-2) obtaining a complementary strand of the corresponding DNA based on the selected sgRNA; adding an AAAC on the 5′ end of the corresponding DNA sequence and adding a C on a 3′ end, so as to obtain a reverse oligo of 5′-AAAC-(20N complementary sequence)-C-3′; and
2-1-3) synthesizing the forward oligo and the reverse oligo respectively.

7. The method, as recited in claim 5, wherein the step 2-3) specifically comprises steps of:
2-3-1) annealing a phosphorylated product of the forward oligo and the reverse oligo, so as to generate fragments with the BsmBI cohesive end; and
2-3-2) ligating the fragments to the lentiCRISPR to form the CRISPR-Cas9 recombinant lentivirus vector.

8. A sgRNA of a human RSPO2 gene targeted with CRISPR-Cas9 specificity, wherein a sgRNA sequence is SEQ ID NO: 2, 4, 6, 8 or 10.

9. A vector containing a DNA sequence corresponding to the sgRNA, as recited in claim 8, wherein the vector is a lentiviral expression vector or not a lentiviral expression vector, which is connected to the DNA sequence

10. The vector containing a DNA sequence corresponding to the sgRNA, as recited in claim 9, wherein the vector is a CRISPR-Cas9 recombinant lentivirus vector.

11. Kits or medicines of the vector recited in claim 9 for treating liver fibrosis.


SEQUENCE LISTING (40 sequences)

SEQ ID NO: 1 - DNA, 23 bp, Artificial Sequence
  RSPO2 target 1 nucleotide sequence
ttgtcttgttcaaaggacaatgg

SEQ ID NO: 2 - RNA, 20 nt, Artificial Sequence
  RSPO2 target 1 sgRNA sequence
uugucuuguucaaaggacaa

SEQ ID NO: 3 - DNA, 23 bp, Artificial Sequence
  RSPO2 target 2 nucleotide sequence
tgtcttgttcaaaggacaatggg

SEQ ID NO: 4 - RNA, 20 nt, Artificial Sequence
  RSPO2 target 2 sgRNA sequence
ugucuuguucaaaggacaau

SEQ ID NO: 5 - DNA, 23 bp, Artificial Sequence
  RSPO2 target 3 nucleotide sequence
ggtgtccatagtacccggatggg

SEQ ID NO: 6 - RNA, 20 nt, Artificial Sequence
  RSPO2 target 3 sgRNA sequence
gguguccauaguacccggau

SEQ ID NO: 7 - DNA, 23 bp, Artificial Sequence
  RSPO2 target 4 nucleotide sequence
cggtgtccatagtacccggatgg

SEQ ID NO: 8 - RNA, 20 nt, Artificial Sequence
  RSPO2 target 4 sgRNA sequence
cgguguccauaguacccgga

SEQ ID NO: 9 - DNA, 23 bp, Artificial Sequence
  RSPO2 target 5 nucleotide sequence
ggctcggtgtccatagtacccgg

SEQ ID NO: 10 - RNA, 20 nt, Artificial Sequence
  RSPO2 target 5 sgRNA sequence
ggcucgguguccauaguacc

SEQ ID NO: 11 - DNA, 23 bp, Artificial Sequence
  RSPO2 target 6 nucleotide sequence
atctgttcatatctggggctcgg

SEQ ID NO: 12 - RNA, 20 nt, Artificial Sequence
  RSPO2 target 6sgRNA sequence
aucuguucauaucuggggcu

SEQ ID NO: 13 - DNA, 23 bp, Artificial Sequence
  RSPO2 target 7 nucleotide sequence
tgtatcaaatcccatttgcaagg

SEQ ID NO: 14 - RNA, 20 nt, Artificial Sequence
  RSPO2 target 7 sgRNA sequence
uguaucaaaucccauuugca

SEQ ID NO: 15 - DNA, 23 bp, Artificial Sequence
  RSPO2 target 8 nucleotide sequence
gtatcaaatcccatttgcaaggg

SEQ ID NO: 16 - RNA, 20 nt, Artificial Sequence
  RSPO2 target 8 sgRNA sequence
guaucaaaucccauuugcaa

SEQ ID NO: 17 - DNA, 23 bp, Artificial Sequence
  RSPO2 target 9 nucleotide sequence
agaacaacttctgttgacatcgg

SEQ ID NO: 18 - RNA, 20 nt, Artificial Sequence
  RSPO2 target 9 nucleotide sequence
agaacaacuucuguugacau

SEQ ID NO: 19 - DNA, 23 bp, Artificial Sequence
  RSPO2 target 10 nucleotide sequence
ggcgcatcccttctcttcgaagg

SEQ ID NO: 20 - RNA, 20 nt, Artificial Sequence
  RSPO2 target 10 sgRNA sequence
ggcgcaucccuucucuucga

SEQ ID NO: 21 - DNA, 23 bp, Artificial Sequence
  RSPO2 target 11 nucleotide sequence
tcttcttccttcgaagagaaggg

SEQ ID NO: 22 - RNA, 20 nt, Artificial Sequence
  RSPO2 target 11 sgRNA sequence
ucuucuuccuucgaagagaa

SEQ ID NO: 23 - DNA, 23 bp, Artificial Sequence
  RSPO2 target 12 nucleotide sequence
gcctgcattcctgcccatccggg

SEQ ID NO: 24 - RNA, 20 nt, Artificial Sequence
  RSPO2 target 12 sgRNA sequence
gccugcauuccugcccaucc

SEQ ID NO: 25 - DNA, 23 bp, Artificial Sequence
  RSPO2 target 13 nucleotide sequence
tgcacatctgttcatatctgggg

SEQ ID NO: 26 - RNA, 20 nt, Artificial Sequence
  RSPO2 target 13 sgRNA sequence
ugcacaucuguucauaucug

SEQ ID NO: 27 - DNA, 23 bp, Artificial Sequence
  RSPO2 target 14 nucleotide sequence
cgaaactgcatctgggcggtcgg

SEQ ID NO: 28 - RNA, 20 nt, Artificial Sequence
  RSPO2 target 14 sgRNA sequence
cgaaacugcaucugggcggu

SEQ ID NO: 29 - DNA, 23 bp, Artificial Sequence
  RSPO2 target 15 nucleotide sequence
aaggcgaaactgcatctgggcgg

SEQ ID NO: 30 - RNA, 20 nt, Artificial Sequence
  RSPO2 target 15 sgRNA sequence
aaggcgaaacugcaucuggg

SEQ ID NO: 31 - DNA, 23 bp, Artificial Sequence
  RSPO2 target 16 nucleotide sequence
gaaaaggcgaaactgcatctggg

SEQ ID NO: 32 - RNA, 20 nt, Artificial Sequence
  RSPO2 target 16 sgRNA sequence
gaaaaggcgaaacugcaucu

SEQ ID NO: 33 - DNA, 23 bp, Artificial Sequence
  RSPO2 target 17 nucleotide sequence
gttcagaatgatgagggcaaagg

SEQ ID NO: 34 - RNA, 20 nt, Artificial Sequence
  RSPO2 target 17 sgRNA sequence
guucagaaugaugagggcaa

SEQ ID NO: 35 - DNA, 23 bp, Artificial Sequence
  RSPO2 target 18 nucleotide sequence
catgcagttcagaatgatgaggg

SEQ ID NO: 36 - RNA, 20 nt, Artificial Sequence
  RSPO2 target 18 sgRNA sequence
caugcaguucagaaugauga

SEQ ID NO: 37 - DNA, 23 bp, Artificial Sequence
  RSPO2 target 19 nucleotide sequence
ccatgcagttcagaatgatgagg

SEQ ID NO: 38 - RNA, 20 nt, Artificial Sequence
  RSPO2 target 19 sgRNA sequence
ccaugcaguucagaaugaug

SEQ ID NO: 39 - DNA, 23 bp, Artificial Sequence
  RSPO2 target 20 nucleotide sequence
cctcatcattctgaactgcatgg

SEQ ID NO: 40 - RNA, 20 nt, Artificial Sequence
  RSPO2 target 20 sgRNA sequence
ccucaucauucugaacugca